(19) United States
(12) Patent Application Publication
(10) Pub. No.: US 2022/0087104 A1
(43) Pub. Date: Mar. 24, 2022

(54) Saffron collecting device

(71) Applicant: STORP AB, MALMÖ (SE)

(72) Inventor: Amir Mehrdad MAHDJOUBI NAMIN, MALMÖ (SE)

(21) Appl. No.: 17/419,507

(22) PCT Filed: Jan. 14, 2020
(86) PCT No.: PCT/SE2020/050028

§ 371 Date: Jun. 29, 2021

(30) Foreign Application Priority Data
Jan. 15, 2019 (SE) 1950037-0

(51) Int. Cl. A01D 46/30; G01N 33/00; A01D 91/04; A01B 1/16

(52) U.S. Cl. CPC A01D 46/30; A01B 1/16; A01D 91/04; G01N 33/0098

(57) ABSTRACT

The present invention describes a saffron collecting device (1) comprising—a sensor unit (2) arranged for identifying pistils of saffron flowers; —a pick unit (3) arranged for removal of the pistils or part thereof from the saffron flowers when the sensor unit (2) has identified the location of the pistils; and —a collector unit (4) arranged for collecting the pistils or part thereof in a collecting space (5).

### FIELD OF THE INVENTION

The present invention relates to a saffron collecting device.

### TECHNICAL BACKGROUND

There are some existing devices intended to simplify the harvesting of saffron. As an example, in CN107278513 there is disclosed an air-suction brush type saffron harvesting device. The saffron harvesting device is said to enable to achieve filament feeding and mechanical harvesting of the saffron seed balls in a natural growth state, and the working efficiency is said to be improved, and labor is liberated. Moreover, in CN107306592 there is disclosed a similar saffron harvesting device. Furthermore, in CN107660380 there is disclosed a pneumatic type saffron filament collection system. Also in this case there is a negative-pressure fan to enable automatic feeding of filaments on saffron seed balls.

The present invention is directed to the provision of an optimal saffron harvesting and collecting device which reduces the labor needed, but which at the same type provides a high yield and high flexibility.

### SUMMARY OF THE INVENTION

The stated purpose above is achieved by a saffron collecting device comprising
a sensor unit arranged for identifying pistils of saffron flowers;
a pick unit arranged for removal of the pistils or part thereof from the saffron flowers when the sensor unit has identified the location of the pistils; and
a collector unit arranged for collecting the pistils or part thereof in a collecting space.

The saffron collecting device operates by use of a sensor unit for the identifying of the pistils of the saffron flowers. Already in this regard, the saffron collecting device according to the present invention differs substantially from the ones disclosed in CN107278513, CN107306592 and CN107660380. The device according to the present invention is first and foremost directed to identifying the pistils of the saffron flowers, and subsequently to that to enable a simple and effective collecting principle of these pistils.

Moreover, in relation to the expression “pistil” it should be understood that this also embodies alternatives to this expression, such as “stigma (of the pistil)” or “stamen”.

### SPECIFIC EMBODIMENTS OF THE INVENTION

Below some specific embodiment of the present invention are disclosed.

According to one embodiment, the collector unit is a bag. The type of bag may vary, but e.g. the type used as dust bags is one possible alternative.

According to one specific embodiment of the present invention, the sensor unit is a camera, IR sensor or radar sensor. Also combinations are possible according to the present invention. Moreover, also combinations of different sensors are possible according to the present invention. According to one preferred embodiment of the present invention, the sensor unit is a camera. As is clear from below, the saffron collecting device according to the present invention may comprise at least two camera units. Therefore, according to one specific embodiment of the present invention, the saffron collecting device comprises at least two sensor units, such as e.g. at least two camera units.

Moreover, according to one embodiment of the present invention, the saffron collecting device comprises at least one sensor unit, preferably a camera unit, for flower detection, and at least one sensor unit, preferably a camera unit, for detection of pistil(s) within the saffron flower. One such example is further explained below.

In this regard, the expression camera embodies any type of camera, such as a CCD (charge-coupled device) or a digital camera. Furthermore, the saffron collecting device according to the present invention may comprise several camera units and sensors, such as one camera unit, e.g. a multi-mono camera unit, for ensuring tracking to find a flower, and one or more camera units, e.g. a stereo pair camera unit, for the tracking within a specific saffron flower to find the pistils/stamens/stigmas of that flower. To give one example, a multi-mono camera unit may be arranged in a robot arm to ensure a suitable tracking and movement of the pick unit to the intended flower, and the pair of stereo cameras may be arranged in close proximity or at the pick unit to ensure efficient picking of the pistil or part of the pistil.

Furthermore, as may be understood from below, the sensor unit suitably comprises a sensor that detects color (reflecting light), physical shape (color separation) with visible light or with other electromagnetic radiation.

In CN107637279 there is disclosed a safflower picking robot. The present invention provides a device with, inter alia, another type of set-up in relation to the implemented sensor(s) and camera(s). Furthermore, the actual tracking technology used, suitably based on color, and the picking sequence, e.g. as explained below, and technology therefore also differ when comparing the present invention with CN107637279. These and other differences will become clearer when reviewing the description below.

Furthermore, according to one embodiment of the present invention, the pick unit is a suction unit, such as a filament suction unit. This may function in different ways, but a negative air pressure is suitable as an operation principle. It should, however, in this regard be noted that also using a negative air pressure in harvesting or a positive air pressure in releasing to a storage container is totally possible according to the present invention.

According to the present invention, negative air pressure does not have to be the only means to remove and collect the pistils once they have been identified. According to one specific embodiment of the present invention, the saffron collecting device also comprises a clip unit arranged to press the suction unit together. The clip unit may be arranged at different places in the device, and may also be part of the suction unit. According to one specific embodiment, the clip unit is arranged onto the suction unit and is programmed to press the same together once a pistil is located inside of the suction unit. The pistil is then fixated securely inside of the suction unit. By moving the suction unit upwards, the pistil is then loosened from the saffron flower and the negative air pressure in the suction unit enables the pistil to be sucked into the collector unit. This is further disclosed below in reference to the method according to the present invention.

It is of course possible according to the present invention to work only based on negative air pressure or vacuum, i.e. without a clip unit. In such a case, however, there is a risk that the vacuum has to be so strong so that also other items may be sucked up into the suction unit. The incorporation of a clip unit may avoid such a potential problem. Moreover, the suction unit may e.g. be made of a compressible material, e.g. a silicone material. This is also suitable so that a regular clip is enabled to press together the suction unit.

According to yet another specific embodiment, the pick unit and the sensor unit are integrated in a 3D unit. Such a 3D unit can be of different type, but may be a device that moves the suction unit and/or camera unit, e.g. one or more digital cameras, around in a harvesting area. For instance, a principle such as a 3D printer may be used also according to the present invention. As mentioned above, the sensor unit may be a camera. In line with this, according to on specific embodiment of the present invention, the sensor unit is a camera unit programmed to identify the pistils based on color. As the pistils have a distinct red color and the flowers are purple, the camera unit may be programmed to identify the pistils based on color and then enable the selection of these pistils to be picked by the pick unit.

One possible configuration of a 3D unit with camera unit(s) are discussed below in terms of functionality. The 3D technology enables the device to localize items in the 3D room, which in itself may be combined with the possibility of moving around in itself (consider mobile version of the device vs a fixed device). One or more camera units working in the visible light range enable to find and localize the pistils based on color. By using digital color filters and optical image processing the camera unit(s) can localize pistils. Moreover, according to one specific embodiment at least two camera sensors may be incorporated in the device according to the present invention. The two units may then operate together based on stereo positioning and triangulation. The position of each camera unit may then be calibrated. Images may then be used to further iterate the procedure until one specific pistil is located. As color filters are set, the entire procedure iterates until the specific color or color range of pistils of saffron flowers are detected.

Based on what has been described above, the area of color filtration and detection may be incorporated into the present invention independently on the camera configuration. Moreover, possible stereo and triangulation methods used may be set according to regular standards. The possible specific adjustment in this technical application may be set into the specific algorithm based on the intended target, i.e. pistils in saffron flowers. This implies that the programming incorporated into the control system of the saffron collecting device should be adapted in accordance with this intended target area.

As may be understood from above, the saffron collecting device suitably operates by means of a processor unit. The technology type may differ, but examples are where the processor unit is a cartesian product or a delta product, e.g. a delta servo product. A delta type is preferred according to the present invention.

The saffron collecting device according to the present invention may be a fixed system or a mobile system. It should, however, be noted that the present invention is in particular related to a mobile system, such as an automated device, such as a robotic device. In the case of a fixed system then saffron flowers have to be harvested and collected first and then emptied or fed to the saffron collecting device according to the present invention. With a mobile saffron collecting device according to the present invention it is possible to obtain the saffron pistils directly from the flowers without first harvesting the same. As may be understood, the mobile saffron collecting device according to the present invention is preferred. In this case, the device performs the entire operation directly on the flowers in an automatic way, without the need for a harvesting operation first. In such a case, one may say that the saffron collecting device also operates as a saffron harvesting and collecting device.

In relation to harvesting and the present invention it may also be said that when only harvesting the pistils the amount of biomass that are removed is relatively small and therefore the need of added fertilizers is low.

In the case of a fixed system, then the saffron collecting device may be arranged in connection to a feeding belt intended for saffron flowers. As such, harvested flowers may be directly put onto this feeding belt.

A mobile device according to the present invention may, with reference to its propulsion principle, be compared to a robotic lawn-mover. The device according to the present invention must also be able to identify the pistils of the saffron flowers. In line with the general description above, according to one specific embodiment of the present invention, the saffron collecting device comprises an automatic propulsion unit and thus is a saffron harvesting and collecting robotic device. Furthermore, according to yet another specific embodiment, the saffron harvesting and collecting robotic device comprises propulsion belts. Moreover, according to one specific embodiment of the present invention, the saffron harvesting and collecting robotic device is arranged with an open space for the sensor unit, such as a camera, and collector unit to be able to detect and pick pistils in growing saffron flowers.

The present invention is also directed to a method for identifying and collecting pistils of saffron flowers. Therefore, according to one embodiment, the present invention refers to a method for identifying and collecting pistils of saffron flowers, wherein a saffron collecting device according to the present invention performs the following operations:
locating a pistil of a saffron flower by means of the sensor unit;
directing a pick unit to the pistil;
removing the pistil from the saffron flower by means of the pick unit; and
directing the pick unit to drop or send the pistil for collecting the pistil in the collector unit.

The operation as such in terms of detection of pistils has been discussed above in general terms.

According to one embodiment of the present invention, the pick unit is a suction unit and where an open end part of the suction unit encloses at least part of the pistil, a clip unit is arranged to then squeeze together said end part of the suction unit to fixate the suction unit around the pistil in a tight way, the suction unit is then directed upwards to loosen or pull the pistil up from the saffron flower and finally the clip unit releases and a negative air pressure allows the suction unit to suck the pistil into the collector unit for collecting the same in the collecting space. As may be understood from above, according to this embodiment the pistil is already loosened when it is finally sucked up into the collector unit. This further implies that the saffron collecting device may have a set negative air pressure which is enough to suck such fixated and loosened pistils, however not loosen the pistils from the flower as such.

It should be noted that the sequence of the method may be performed in different ways. The following is another example how the procedure may be performed. First the camera unit identifies saffron flowers and provides X, Y and Z coordinates to each flower cluster inside of the working area of the picking area. The X coordinate is changed when the robot device moves in one direction. When the X coordinate is the same as the center of the flower cluster, then the pick unit is moved to the right Y coordinate. As mentioned below, the sensor unit may comprise several camera units. For instance, a single or double stereo camera units may take over the next step in the operation sequence once the right y coordinate has been set. The stereo cameras then control the pick unit to be driven to the top of a pistil of the saffron flower. This procedure may be accomplished based on color identification in the software used. The procedure above may be seen as an effective way of ensuring that the pick unit is ensured to be arranged in the right place before starting the actual picking procedure.

As a next step, the pick unit is opened and vacuum is provided so that the pick unit is in a sucking mode. The pick unit then is moved to the root of the pistil and is closed. The pick unit is then moved in a Z coordinate direction to loosen the pistil. Once the movement is finalized then the pick unit releases its grip of the pistil. The vacuum provided ensures that the pistil is sucked into the collector unit, such as a bag or container or the like. The pick unit moves to the next pistil top and at the same time the pick unit e.g. closes once the earlier pistil is detected in the collector unit. Repetition is performed of the actual picking sequence until all pistils in the flower cluster has been picked. Then movement is made to the next flower cluster, such as described in the beginning of the sequence.

It should be noted that the above is explaining a sequence in a dry condition. In wet, mist or rain, a pick and place method is needed. Suction to harvest and a blow out when a pick unit head has moved to the collecting point are suitably incorporated.

The present invention also refers to a related type of device intended to remove the problem of weed around the saffron flowers after harvesting. The harvesting time is normally just in the range of 2 weeks during the year. After that it is important to remove weed to enable for next year's growth and harvest. If not, then it is not possible to grow new flowers at the same growth rate in an intended way during next year. The concept according to the present invention may also be used for this purpose. Therefore, according to one specific embodiment of the present invention, there is provided a weed removing device comprising:
a sensor unit arranged for identifying weed around low growing plants; and
a pressing unit arranged for pressing the weed into the ground when the sensor unit has identified the location of the weed.

Also in this case, the device may operate on the same principle as the saffron collecting device. For instance, the sensor unit may comprise one or more camera units which may operate based on color. In this case, the color filter should e.g. be operating in the green visible field so that all green items except the leaves of saffron flowers are pushed down into the ground. Therefore, according to one specific embodiment of the present invention, the sensor unit is a camera sensor and is arranged to identify the weed based on color. The arrangement may be based on triangulation and/or the implementation of color filters in the software. For example, green parts not extending more than a certain range above ground is programmed to be pressed down into the ground. In such case, then both triangulation and color filters should be used in the control mechanism. In another case then only a color filter may be implemented. It should be possible to distinguish leaves of the saffron flowers, which are needed for next year's growth of saffron flowers, from weed around such leaves. If a color filter is set where such separation may be made, then only a color filter may be implemented.

Moreover, the lower part of the saffron flower is pale, which also may be used for identification reasons.

Also 3D technology is suitable to implement in the weed removing device according to the present invention.

It may be mentioned that one robotic vehicle for weed treatment is disclosed in WO2017/002093. The robotic vehicle is arranged for carrying out an automatic treatment method comprising the steps of capturing images of a weed with a camera on said robot, determining with said images a distance between said weed and a cultivated plant, and then selecting a treatment tool from a group of treatment tools of said robot for a treatment of the weed as function of said distance. The robotic vehicle comprises an image acquisition device for capturing images, a computer vision system arranged for determining from said images a distance between a weed and a cultivated plant; a selecting system arranged for selecting a treatment tool from a group of treatment tools for a treatment of the weed as function of said distance; and a tools supporting system arranged to support the treatment tools and arranged to move at least operational extremities of said treatment tools with respect to the robot vehicle.

As should be obvious from above, the device and method according to WO2017/002093 is directed to treatment of weed, and not for collecting saffron or handling saffron plants after harvesting. The device according to WO2017/002093 does not comprise a pressing unit being part of the weed removing device according to one embodiment of the present invention.

Moreover, it is according to the present invention possible to integrate the capability of removing weed according to above in a saffron collecting device according to the present invention. In such a case, the specific saffron collecting device has both capabilities, that is programmed to collect pistils, and also programmed to detect weed around the saffron flowers and push them down into the ground.

### DETAILED DESCRIPTION OF THE DRAWINGS

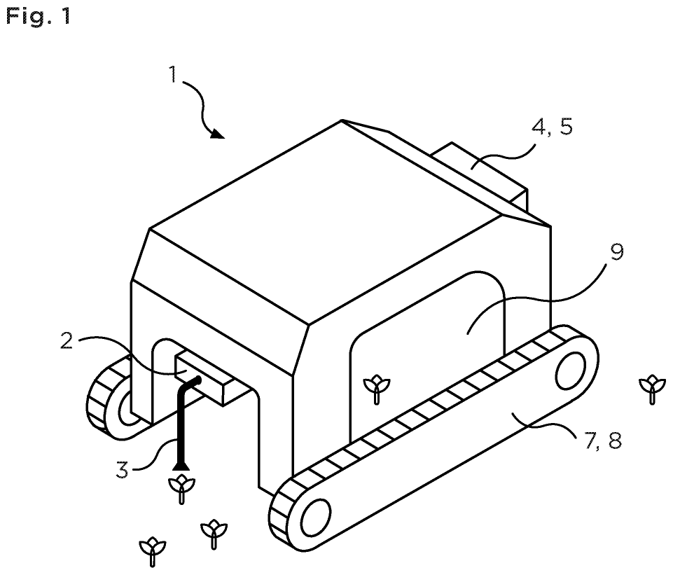

In FIG. 1 there is shown one embodiment of the present invention. In this case the saffron collecting device 1 is in the form of a mobile version, such as a robotic self-transporting saffron collecting device 1. The saffron collecting device 1 comprises a sensor unit 2, which suitably comprises one or more camera units. Moreover, the saffron collecting device 1 also comprises a pick unit arranged for removal of the pistils or part thereof from the saffron flowers when the sensor unit 2 has identified the location of the pistils. In this case the sensor unit 2 and the pick unit 3 are integrated in one and the same 3D unit. Moreover, in this case the pick unit 3 is in the form of a suction unit which enables to suck up the pistils of the saffron flowers. A collector unit 4 is arranged for collecting the pistils or part thereof in a collecting space 5. This may be a box which comprises a bag being connected to the suction unit, similar to the concept of a vacuum cleaner. Furthermore, according to this embodiment, the saffron collecting device 1 comprises an automatic propulsion unit 7, i.e. the device 1 is a saffron harvesting and collecting robotic device, with propulsion belts 8 in this case. Furthermore, the saffron harvesting and collecting robotic device 1 is arranged with an open space 9 for the camera unit and collector unit to be able to detect and pick pistils in growing saffron flowers in an effective way.

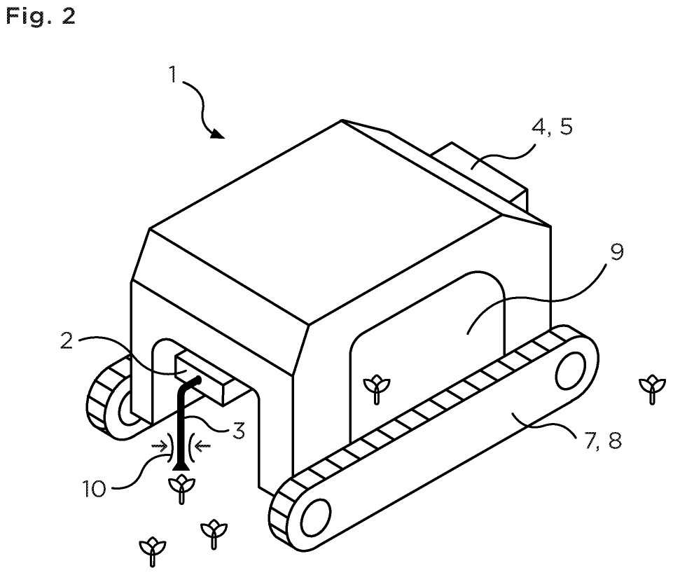

In FIG. 2 there is shown a similar embodiment as in FIG. 1, however in this case the device 1 also comprises a clip unit 10. This clip unit 10 is part of the 3D unit with sensor unit and pick unit, i.e. suction unit. The clip is arranged to enable to squeeze the suction unit so that an enclosed pistil may be pulled up and loosened from a saffron flower.

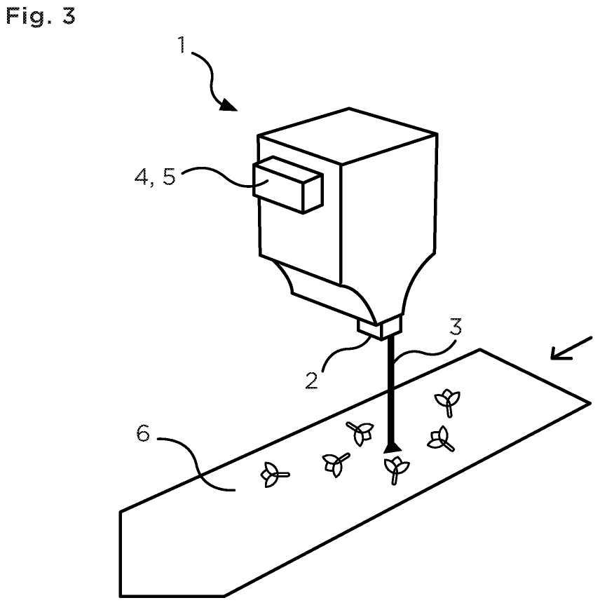

In FIG. 3 there is shown another embodiment of the present invention. In this case, the device 1 is a fixed device. Therefore, the saffron collecting device 1 is arranged in connection to a feeding belt 6 intended for saffron flowers. Otherwise, also in this case the saffron collecting device 1 comprises a sensor unit 2, which suitably comprises one or more camera units. Moreover, the saffron collecting device 1 also comprises a pick unit arranged for removal of the pistils or part thereof from the saffron flowers when the sensor unit 2 has identified the location of the pistils. Also in this case the sensor unit 2 and the pick unit 3 are integrated in one and the same 3D unit. Moreover, also in this case the pick unit 3 is in the form of a suction unit which enables to suck up the pistils of the saffron flowers and collect the pistils in a collector unit 4 connected to a collecting space 5.

1. A saffron collecting device comprising
a sensor unit arranged for identifying pistils of saffron flowers;
a pick unit arranged for removal of the pistils or part thereof from the saffron flowers when the sensor unit has identified the location of the pistils; and
a collector unit arranged for collecting the pistils or part thereof in a collecting space.

2. The saffron collecting device according to claim 1, wherein the sensor unit is a camera, IR sensor or radar sensor, preferably a camera.

3. The saffron collecting device according to claim 1, wherein the saffron collecting device comprises at least two sensor units.

4. The saffron collecting device according to claim 1, wherein the saffron collecting device comprises at least one sensor unit for flower detection, and at least one sensor unit for detection of pistil(s) within the saffron flower.

5. The saffron collecting device according to claim 1, wherein the pick unit is a suction unit.

6. The saffron collecting device according to claim 5, wherein the saffron collecting device also comprises a clip unit arranged to press the suction unit together.

7. The saffron collecting device according to claim 1, wherein the pick unit and the sensor unit are integrated in a 3D unit.

8. The saffron collecting device according to claim 1, wherein the sensor unit is a camera unit programmed to identify the pistils based on color.

9. The saffron collecting device according to claim 1, wherein the saffron collecting device operates by means of a processor unit.

10. The saffron collecting device according to claim 9, wherein the processor unit is a cartesian product or a delta servo product.

11. The saffron collecting device according to claim 1, wherein the saffron collecting device is arranged in connection to a feeding belt intended for saffron flowers.

12. The saffron collecting device according to claim 1, wherein the saffron collecting device comprises an automatic propulsion unit and thus is a saffron harvesting and collecting robotic device.

13. The saffron collecting device according to claim 12, wherein the saffron harvesting and collecting robotic device comprises propulsion belts.

14. The saffron collecting device according to claim 12, wherein the saffron harvesting and collecting robotic device is arranged with an open space for the sensor unit and collector unit to be able to detect and pick pistils in growing saffron flowers.

15. A method for identifying and collecting pistils of saffron flowers, wherein a saffron collecting device according to claim 1 performs the following operations:
locating a pistil of a saffron flower by means of the sensor unit;
directing a pick unit to the pistil;
removing the pistil from the saffron flower by means of the pick unit; and
directing the pick unit to drop or send the pistil for collecting the pistil in the collector unit.

16. The method according to claim 15, wherein the pick unit is a suction unit and wherein an open end part of the suction unit encloses at least part of the pistil, a clip unit is arranged to then squeeze together said end part of the suction unit to fixate the suction unit around the pistil in a tight way, the suction unit is then directed upwards to loosen or pull the pistil up from the saffron flower and finally the clip unit releases and a negative air pressure allows the suction unit to suck the pistil into the collecting unit for collecting the same in the collecting space.

17. A weed removing device comprising:
a sensor unit arranged for identifying weed around low growing plants; and
a pressing unit arranged for pressing the weed into the ground when the sensor unit has identified the location of the weed.

18. The weed removing device according to claim 17, wherein the sensor unit is a camera sensor and is arranged to identify the weed based on color.
